(19) United States
(12) Patent Application Publication
(10) Pub. No.: US 2015/0018760 A1
(43) Pub. Date: Jan. 15, 2015

(54) NASAL IRRIGATION SOLUTION PREPARATION SYSTEM AND METHOD OF PREPARATION

(71) Applicants: Kathryn Morton HARDIN, Charlotte, NC (US); James Craig HARDIN, Charlotte, NC (US)

(72) Inventors: Kathryn Morton HARDIN, Charlotte, NC (US); James Craig HARDIN, Charlotte, NC (US)

(21) Appl. No.: 14/329,135

(22) Filed: Jul. 11, 2014

Related U.S. Application Data
(60) Provisional application No. 61/844,955, filed on Jul. 11, 2013.

(51) Int. Cl. A61J 1/20; A61J 1/14; A61M 3/02

(52) U.S. Cl. CPC A61J 1/2093; A61M 3/0254; A61J 1/1443; A61J 2001/1456; A61J 2200/40; A61M 2210/0618
USPC 604/82; 604/257; 604/113

(57) ABSTRACT

A nasal irrigation system for rinsing nasal and sinus passages or cavities includes a solution preparation unit and a dispensing container. The solution preparation unit includes a first water reservoir, a filter that is in fluid communication with an outlet of the first water reservoir and filters water passing therethrough, a second water reservoir that is in fluid communication with the filter, and a heating element that brings filtered water in the second water reservoir to a boil to clean the filtered water, and a filler port. The dispensing container receives a nasal rinse solution from the solution preparation unit, via the filler port, and administers the nasal rinse solution to a user via a direct nostril interface. The solution preparation unit can also include a cooling element, additive reservoirs for materials to calibrate salinity or to provide a pH buffer, and a control circuit to control operation thereof.

### CROSS-REFERENCE TO RELATED APPLICATION

The present application is a U.S. nonprovisional patent application of, and claims priority under 35 U.S.C. §119(e) to, U.S. provisional patent application Ser. No. 61/844,955, filed Jul. 11, 2013, which provisional patent application is incorporated by reference herein.

### COPYRIGHT STATEMENT

All of the material in this patent document is subject to copyright protection under the copyright laws of the United States and other countries. The copyright owner has no objection to the facsimile reproduction by anyone of the patent document or the patent disclosure, as it appears in official governmental records but, otherwise, all other copyright rights whatsoever are reserved.

### BACKGROUND OF THE PRESENT INVENTION

1. Field of the Present Invention

The present invention relates generally to a nasal irrigation system and, in particular, to a nasal irrigation system for use in rinsing a user's nostrils and sinus passages.

2. Background

Nasal and sinus complaints are common clinical problems with a negative impact on the quality of life for those who are afflicted. Main categories of nasal and sinus problems include inflammation of the paranasal sinuses; upper respiratory infections; and allergic rhinitis. Rinsing the nasal and sinus cavities has been practiced for a number of years as an effective way for a person afflicted by nasal or sinus problems to experience relief from discomfort. Furthermore, rinsing the nasal and sinus passages has the beneficial effect of treating congested sinuses, moistening the nasal passages, flushing pathogens, and removing foreign irritants.

Currently there are several variations of nasal rinsing devices on the market. However, the current nasal rinsing devices on the market are considered to be time consuming and difficult to prepare. The Food and Drug Administration has issued warnings about the source and quality of water that is used with nasal rinsing devices. As specified by the Food and Drug Administration, tap water that is not filtered, treated, or processed in specific ways is not safe for use as a nasal rinse. Unfortunately, it can be tedious, time-consuming and/or inconvenient to prepare water properly. As a result, a person afflicted by the discomfort of nasal or sinus problems may choose to forgo using a nasal rinsing device because of the inconvenience.

Accordingly, a need exists for a safe nasal and sinus rinse capable of being prepared in a convenient manner. These, and other needs, are addressed by one or more aspects of the present invention.

### SUMMARY OF THE PRESENT INVENTION

Broadly defined, the present invention is a nasal irrigation system for rinsing nasal and sinus passages or cavities, including: a solution preparation unit, including a purified water reservoir, a base, and a filler port disposed above and over the base, that dispenses purified water through the filler port for use in a nasal rinse solution; and a dispensing container that receives the nasal rinse solution, while positioned on the base, from the filler port of the solution preparation unit, and that administers the nasal rinse solution, while removed from the base, to a user via a direct nostril interface.

In a feature of this aspect, the purified water reservoir is disposed within a housing that is supported by above the base. In further features, the nasal irrigation system further includes a control circuit; the nasal irrigation system further includes a second water reservoir and a filter, the second water reservoir being in fluid communication with the filter and the purified water reservoir such that water contained in the second water reservoir flows from an outlet, through the filter, and into the purified water reservoir; the filter is a particle filter having a pore size of one-micron or less; the nasal irrigation system further includes a heating element, under the control of the control circuit, that is in thermal contact with the purified water reservoir; the heating element is adapted to boil water contained in the purified water reservoir; and/or the heating element is adapted to maintain water contained in the purified water reservoir at a temperature of 85-90 degrees Fahrenheit.

Broadly defined, the present invention according to another aspect is a nasal irrigation system for rinsing nasal and sinus passages or cavities, including: a solution preparation unit having a first water reservoir, a filter that is in fluid communication with an outlet of the first water reservoir and filters water passing therethrough, a second water reservoir that is in fluid communication with an outlet of the filter, a heating element that brings filtered water in the second water reservoir to a boil to disinfect and sterilize the filtered water, and a filler port; and a dispensing container that receives a nasal rinse solution from the solution preparation unit, via the filler port, and administers the nasal rinse solution to a user via a direct nostril interface. The nasal irrigation system may further include a control circuit.

In a feature of this aspect, the nasal irrigation system further includes a cooling element that cools heated water in the second water reservoir to a set temperature under the control of the control circuit. In a further feature, the cooling element cools heated water in the second water reservoir to a temperature set by a user.

In another feature of this aspect, the heating element maintains water contained in the second water reservoir at a set temperature under the control of the control circuit. In further features, the heating element maintains water contained in the second water reservoir at a temperature set by a user; and/or the heating element maintains water contained in the second water reservoir at a temperature of 85-90 degrees Fahrenheit.

In another feature of this aspect, the nasal irrigation system further includes a pump that forces water from the first water reservoir through the filter to the second water reservoir.

In another feature of this aspect, the water flows from the first water reservoir through the filter to the second water reservoir via gravity flow.

Broadly defined, the present invention according to another aspect is a nasal irrigation system for rinsing nasal and sinus passages or cavities, including: a solution preparation unit, having a water reservoir, a device for purifying water in the water reservoir, an additive reservoir, and a filler port; and a dispensing container that receives a nasal rinse solution from the solution preparation unit, via the filler port, and administers the nasal rinse solution to a user via a direct nostril interface; wherein the nasal rinse solution is a combination of purified water from the water reservoir and an additive from the additive reservoir. The nasal irrigation system may further include a control circuit.

In a feature of this aspect, the device for purifying water is a particle filter that removes particles from water flowing into the water reservoir.

In another feature of this aspect, the device for purifying water is a heating element that brings water in the water reservoir to a boil.

In another feature of this aspect, the additive reservoir contains non-iodized salt to calibrate the salinity of the nasal rinse solution.

In another feature of this aspect, the additive reservoir contains a medicinal or therapeutic material. In a further feature, the medicinal or therapeutic material is an allergenic, analgesic, and/or antibiotic material.

In another feature of this aspect, the additive reservoir contains a pH buffer. In a further feature, the pH buffer is sodium bicarbonate.

Broadly defined, the present invention according to another aspect includes a nasal irrigation system that includes a container for holding and dispensing nasal rinse solution and solution preparation unit for preparation of nasal rinse solution. The container includes an extending effluent spout that is interconnected to the body of the container. The solution preparation unit includes a base and an upper housing. The upper housing further includes a water reservoir to hold water; a filter to receive and filter water from the water reservoir; a filtered-water reservoir to hold filtered water from the filter; and a filler port to dispense a solution of into the container. A passage connects the filtered-water reservoir to the filler port. The filler port dispenses filtered water to the container.

In features of this aspect, nasal irrigation system may be inserted in to the solution preparation unit to rest on the base. In a further feature, the base includes a warming plate and a cooling plate.

In a further feature of this aspect, the irrigant compound comprises non-iodized salt. In a further feature, the irrigant compound is comprised of sodium bicarbonate. In a further feature, the irrigant compound comprises a mixture thereof including non-iodized salt, eucalyptus, chamomile or medicines including allergenic, analgesics, or antibiotics. In a further feature, irrigant compound comprises a mixture thereof including sodium bicarbonate, eucalyptus, chamomile or medicines including allergenics, analgesics, or antibiotics. In yet a further feature, the irrigant compound comprises a mixture thereof including eucalyptus, chamomile and medicines including allergenics, analgesics, or antibiotics.

In another feature of this aspect, the filter has a pore size of one-micron or less to filter particulates from water. In further feature, gravity flow moves water from the water reservoir, through the filter and into the filtered-water reservoir. In yet a further feature, pressure moves water from the water reservoir, through the filter and into the filtered-water reservoir.

In another feature of this aspect, the housing includes a pump that moves water from the water reservoir, through the filter and into the filtered-water reservoir. In a further feature, the pump is activated by an on-control.

In another feature of this aspect, the housing includes a control circuit. In another feature of this aspect, the housing includes a processor having an internal clock with current time output. In a further feature, the control circuit includes an input unit for inputting a present activation and deactivation time.

In another feature of this aspect, the water reservoir is detachable from the housing.

In another feature of this aspect, housing includes a passage way that connects the water reservoir, the filtered-water reservoir, and the least one irrigant compound reservoir to the filler port. In a further feature, the passage way is tubing.

In another feature of this aspect, housing includes housing further includes a start control that releases the filtered water from the filtered-water reservoir. In a further feature, housing includes a start control that release the irrigant compound from the at least one irrigant compound reservoir.

In another feature of this aspect, the housing includes a heating element to heat filtered water contained in the filtered-water reservoir. In a further feature, the heating element heats the filtered water to a boil. In a further feature, the filtered water is boiled for at least three minutes to reduce the presence of microbial agents in the water. In a further feature, the housing includes a cooling element to cool filtered water contained in the filtered-water reservoir. In a further feature, the housing includes a temperature adjustment so the user can select a target temperature. In a further feature, the housing includes a control circuit. In a further feature, the housing includes a processor having an internal clock with current time output. In a further feature, control circuit includes an input unit for inputting a present activation and deactivation time for the heating and cooling elements. In a further feature, the housing includes a portion of a remote control system configured for remote communication and providing command options for interaction with the processor. In a further feature, the remote control system is usable with a tablet. In yet a further feature, the remote control system is usable with a smart phone.

Broadly defined, the present invention according to another aspect includes a nasal irrigation system as shown and described.

Broadly defined, the present invention according to another aspect includes a nasal irrigation system that conveniently prepares water for nasal rinse. The nasal irrigation system includes a filter that filters water flowing from a non-filtered water reservoir to a filtered-water reservoir, a heating element that brings filtered water in a filtered-water reservoir to a boil to disinfect and sterilize the filtered water, a cooling element that cools water in the filtered-water reservoir to a user selected temperature, and a filler port that releases the filtered water, after the user-selected temperature is achieved, into a container for holding and dispensing nasal rinse solution.

In a feature of this aspect, the filter has a pore size of one-micron or less to filter particulates from water.

In a feature of this aspect, the heating element heats the filtered water to a boil. In a further feature, the filtered water is boiled for at least three minutes to reduce the presence of microbial agents in the water.

In a feature of this aspect, the cooling element includes a coil to constitute a means of transfer heat. In yet a further feature, the cooling element includes at least one fan and vent to cool the filtered water in the filtered-water reservoir by the circulation of air.

Broadly defined, the present invention according to another aspect includes a nasal irrigation system that conveniently prepares water for nasal rinse as shown and described.

Broadly defined, the present invention according to another aspect includes a method of irrigating a user's nostrils and sinus cavities. The method includes providing a nasal irrigation solution preparation system having a dispensing container and a solution preparation unit. The dispenser container includes a handle and an extending effluent spout. The method further includes: filling the dispensing container with an irrigation solution; sealing the nasal passage of a user to the extending effluent spout of the dispensing container; and releasing the irrigation solution from the dispensing container into the passageway and nasal cavity.

In a feature of this aspect, the solution preparation unit includes at least one irrigant compound reservoirs; a first water reservoir; a second water reservoir; a heating unit to heat at least one of the water reservoirs; and a cooling unit to cool at least one of the water reservoirs.

In a feature of this aspect, the irrigate compound reservoir is filled with an irrigate compound.

In a feature of this aspect, the heating unit heats water in the at least one water reservoirs to a boil. In a further feature, cooling unit at cools water in the at least one water reservoirs to a target temperature.

Broadly defined, the present invention according to another aspect includes a method of irrigating a user's nostrils and sinus cavities as shown and described.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

Further features, embodiments, and advantages of the present invention will become apparent from the following detailed description with reference to the drawings, wherein:

FIG. 1 is a front perspective view of a nasal irrigation solution preparation system in accordance with one or more preferred embodiments of the present invention;

FIG. 2 is a partially schematic top view of the upper assembly of the nasal irrigation solution preparation system of FIG. 1;

FIG. 3 is a partially cutaway front view of the nasal irrigation solution preparation system of FIG. 1;

FIG. 4 is a front view of the nasal irrigation solution preparation system of FIG. 1;

FIG. 5 is a front view of the nasal irrigation solution preparation system of FIG. 1 shown with the dispensing container removed from the solution preparation unit; and

FIGS. 6A and 6B are side views of the dispensing container of FIG. 1 showing the dispensing container with the lid attached.

### DETAILED DESCRIPTION

As a preliminary matter, it will readily be understood by one having ordinary skill in the relevant art (“Ordinary Artisan”) that the present invention has broad utility and application. Furthermore, any embodiment discussed and identified as being “preferred” is considered to be part of a best mode contemplated for carrying out the present invention. Other embodiments also may be discussed for additional illustrative purposes in providing a full and enabling disclosure of the present invention. As should be understood, any embodiment may incorporate only one or a plurality of the above-disclosed aspects of the invention and may further incorporate only one or a plurality of the above-disclosed features. Moreover, many embodiments, such as adaptations, variations, modifications, and equivalent arrangements, will be implicitly disclosed by the embodiments described herein and fall within the scope of the present invention.

Accordingly, while the present invention is described herein in detail in relation to one or more embodiments, it is to be understood that this disclosure is illustrative and exemplary of the present invention, and is made merely for the purposes of providing a full and enabling disclosure of the present invention. The detailed disclosure herein of one or more embodiments is not intended, nor is to be construed, to limit the scope of patent protection afforded the present invention, which scope is to be defined by the claims and the equivalents thereof. It is not intended that the scope of patent protection afforded the present invention be defined by reading into any claim a limitation found herein that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present invention. Accordingly, it is intended that the scope of patent protection afforded the present invention is to be defined by the appended claims rather than the description set forth herein.

Additionally, it is important to note that each term used herein refers to that which the Ordinary Artisan would understand such term to mean based on the contextual use of such term herein. To the extent that the meaning of a term used herein—as understood by the Ordinary Artisan based on the contextual use of such term—differs in any way from any particular dictionary definition of such term, it is intended that the meaning of the term as understood by the Ordinary Artisan should prevail.

Regarding applicability of 35 U.S.C. §112, ¶6, no claim element is intended to be read in accordance with this statutory provision unless the explicit phrase “means for” or “step for” is actually used in such claim element, whereupon this statutory provision is intended to apply in the interpretation of such claim element.

Furthermore, it is important to note that, as used herein, “a” and “an” each generally denotes “at least one,” but does not exclude a plurality unless the contextual use dictates otherwise. Thus, reference to “a picnic basket having an apple” describes “a picnic basket having at least one apple” as well as “a picnic basket having apples.” In contrast, reference to “a picnic basket having a single apple” describes “a picnic basket having only one apple.”

When used herein to join a list of items, “or” denotes “at least one of the items,” but does not exclude a plurality of items of the list. Thus, reference to “a picnic basket having cheese or crackers” describes “a picnic basket having cheese without crackers,” “a picnic basket having crackers without cheese,” and “a picnic basket having both cheese and crackers.” Finally, when used herein to join a list of items, “and” denotes “all of the items of the list.” Thus, reference to “a picnic basket having cheese and crackers” describes “a picnic basket having cheese, wherein the picnic basket further has crackers,” as well as describes “a picnic basket having crackers, wherein the picnic basket further has cheese.”

Referring now to the drawings, in which like numerals represent like components throughout the several views, one or more preferred embodiments of the present invention are next described. The following description of one or more preferred embodiment(s) is merely exemplary in nature and is in no way intended to limit the invention, its application, or uses.

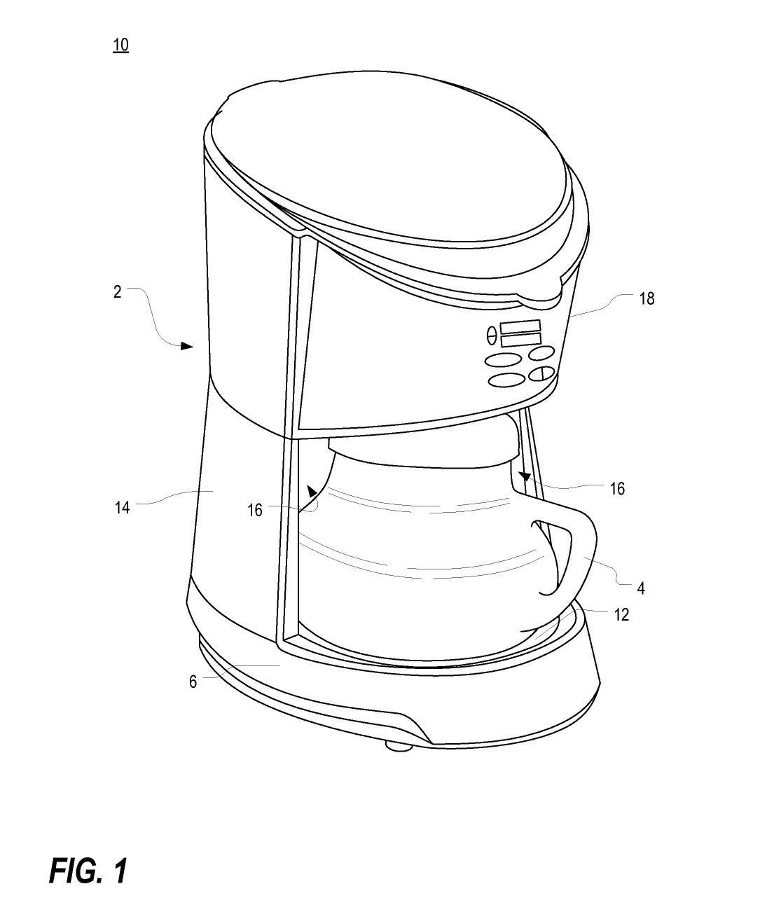

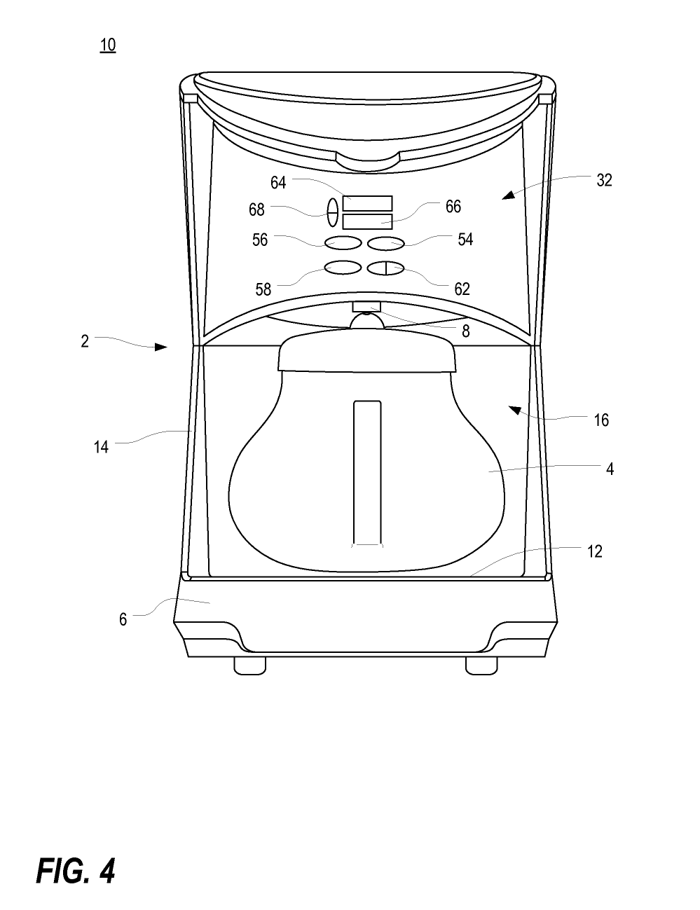

FIG. 1 is a front perspective view of a nasal irrigation solution preparation system 10 in accordance with one or more preferred embodiments of the present invention. As shown in FIG. 1, the nasal irrigation system 10 includes a solution preparation unit 2 and a dispensing container 4. The solution preparation unit 2 has a base 6 on which the dispensing container 4 can be placed beneath a filler port 8 (shown in FIG. 4). In at least one embodiment, the base 6 also includes a warming-cooling plate 12 to help maintain the controlled temperature of the solution in the dispensing container 4. Connected to the base 6 is a “U”-shaped upstanding wall 14 with an open front portion 16 whereby at least a portion of the dispensing container 4 may be inserted to rest on the base 6. The upstanding wall 14 supports an upper assembly 18, described below.

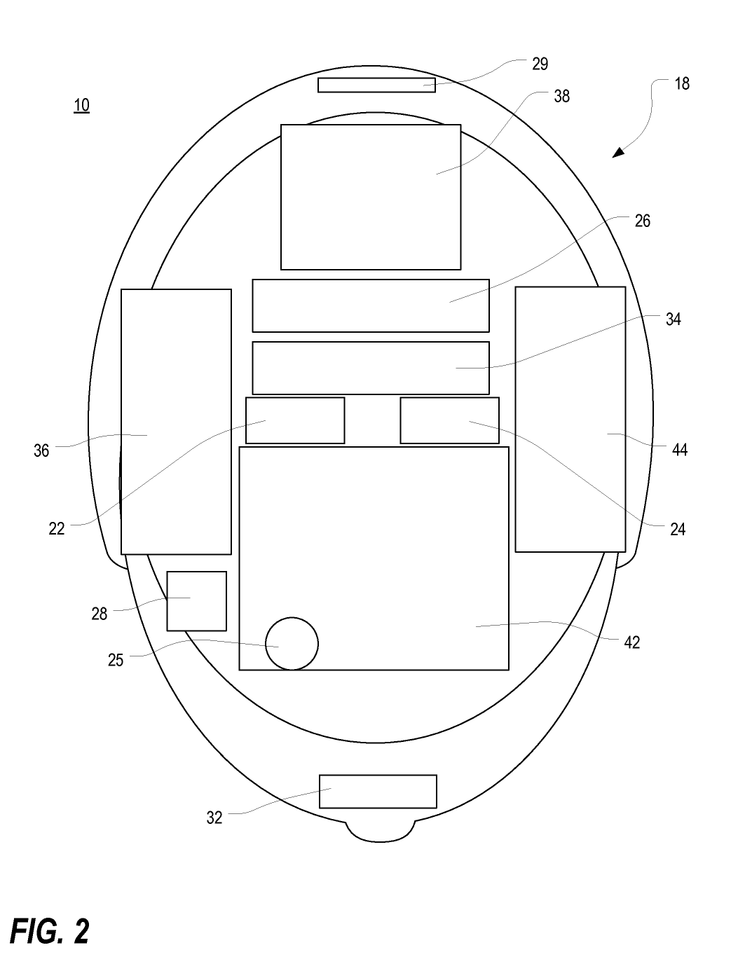

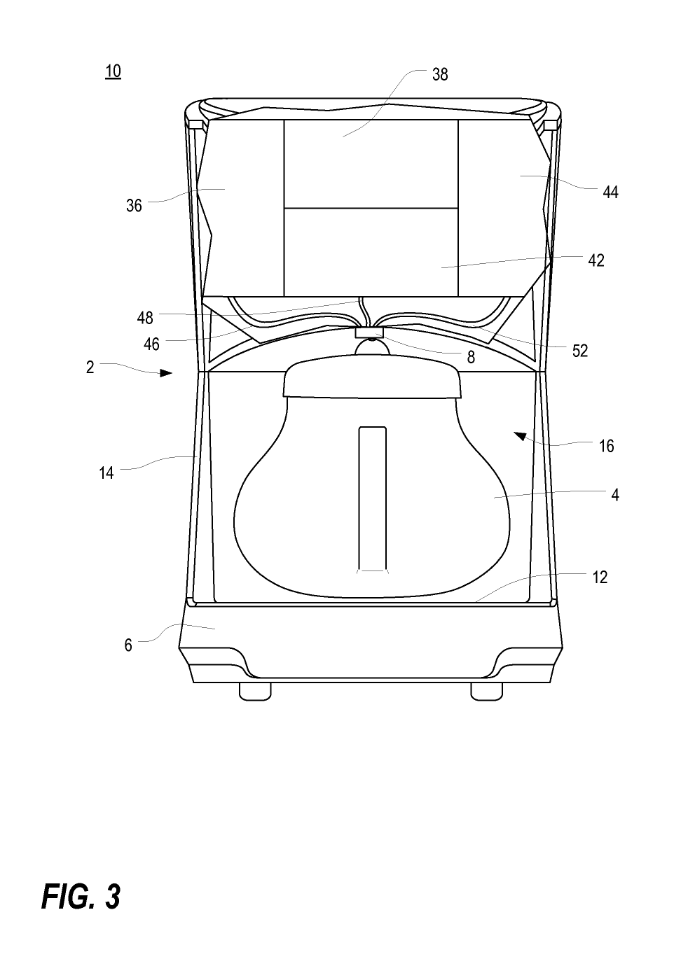

FIG. 2 is a partially schematic top view of the upper assembly 18 of the nasal irrigation system 10 of FIG. 1, and FIG. 3 is a partially cutaway front view of the nasal irrigation system 10 of FIG. 1. As collectively shown therein, the upper assembly 18 may include a heating element 22, a cooling element 24, a temperature sensor 25, a pump 26, a processor 28, a power supply 29, a control panel 32, a filter 34, a pair of water reservoirs 38,42, a pair of additive reservoirs 36,44, and/or a plurality of tubes or other passages 46,48,52 connecting various respective reservoirs 36,42,44, respectively, to the filler port 8. Each of these will be described in greater detail hereinbelow.

The water reservoirs 38,42 include a purified water reservoir 42 and a tap water reservoir 38, wherein the purified water reservoir 42 contains or is adapted to contain a quantity of filtered water and the tap water reservoir 38 contains or is adapted to contain a quantity of tap water. The additive reservoirs may include a non-iodized salt reservoir 36 and a pH buffer reservoir 44, wherein the non-iodized salt reservoir 36 contains or is adapted to contain a quantity of non-iodized salt, and the pH buffer reservoir 44 contains or is adapted to contain a quantity of a pH buffer, such as sodium bicarbonate. Filtered water may, in some embodiments, be added to the purified water reservoir 42 directly, but in at least some embodiments, the filtered water is produced from tap water contained in the tap water reservoir 38, which is preferably detachable from the solution preparation unit 2 for retrieving a source water. In at least one embodiment, the non-iodized salt reservoir 36, the purified water reservoir 42, and the pH buffer reservoir 44 are also each separately detachable from the solution preparation unit 2. The water in the tap water reservoir 38 is filtered through the filter 34 and stored in the purified water reservoir 42. The filter 34, in at least some embodiments, is a one-micron or less ultra-pure filter. In at least one embodiment, the pump 26 is used to force water in the tap water reservoir 38 through the filter 34 to the purified water reservoir 42. In another embodiment, gravity flow is used to force the water in the water reservoir 38 through the filter 34 into the purified water reservoir 42.

As shown in FIG. 3, the non-iodized salt reservoir 36, the purified water reservoir 42, and the pH buffer reservoir 44 are connected to the filler port 8 via passages or tubes 46,48,52. As described further hereinbelow, the solution preparation unit 2 further includes a start control 54 that controls the release, concurrently or non-concurrently, of desired quantities of non-iodized salt, filtered water, and sodium bicarbonate from the respective non-iodized salt reservoir 36, the purified water reservoir 42, and the pH buffer reservoir 44 through the respective passages or tubes 46,48,52 and the filler port 8 into the dispensing container 4.

FIG. 4 is a front view of the nasal irrigation system 10 of FIG. 1. As shown therein, the control panel 32 may include an on-control 56, an off-control 58, a temperature adjustment control 62, a temperature display 64, a time display 66, and/or the start control 54. The heating element 22 and the cooling element 24 are manually activated by the on-control 56 and the off-control 58. In an alternative embodiment, on-off activation is included in a single button. In various other alternative embodiments, multiple functions are controlled via a single button. The on-control 56 activates the heating element 22 and the cooling element 24 to make an electrical connection between the heating and cooling elements 22,24 and the power supply 29. In at least some embodiments, the on-control 56 also activates the pump 26 to make an electrical connection between the pump 26 and the power supply 29. Conversely, the off-control 58 deactivates the heating element 22 and the cooling element 24 to make an electrical disconnection between the heating and cooling elements 22,24 and the power supply 29. In at least some embodiments, the off-control 58 also deactivates the pump 26 to make an electrical disconnection between the pump 26 and the power supply 29. In at least one embodiment, the off-control 58 automatically deactivates the heating element 22 and the cooling element 24 after a period of time set by a user. In another embodiment, the off-control 58 automatically deactivates the heating element 22 and the cooling element 24 after a thirty-minute period or another predefined period.

The heating element 22 is in thermal contact with the purified water reservoir 42 so that water in the purified water reservoir 42 is temperature controlled. The heating element 22 initially brings water in the purified water reservoir 42 to a boil for at least three minutes to reduce or preferably eliminate the presence of microbial agents in the filtered water. Thereafter, in at least one embodiment, the cooling element 24 reduces the temperature of the water in the purified water reservoir 42 to a selected comfort temperature. In at least some embodiments, the cooling element 24 includes a cooling coil immersed in the purified water reservoir 42 to constitute a means of transferring heat. In at least some embodiments, the cooling element 24 includes at least one fan and vent to cool the purified water reservoir 42 via circulation of ambient air to facilitate the dissipation of heat from the purified water reservoir 42. In at least some embodiments the cooling element 24 includes other means of heat transfer to reduce the temperature of the water in the purified water reservoir 42 to a selected comfort temperature. For example, in a further embodiment, the temperature of the water in the purified water reservoir 42 is cooled by contact with ambient air. The temperature of the water in the purified water reservoir 42 may be selected by the user by increasing or decreasing the adjustment control 62 to a target value. The temperature display output 64 indicates the current temperature of the water as measured by the temperature sensor 25 in contact with water in the purified water reservoir 42. When the temperature of the filtered water measures below the specified target value, the heating element 22 raises the temperature of the water in the purified water reservoir 42 to the specified target value. When the temperature of the filtered water measures above the specified target value, the cooling element 24 lowers the temperature of the water in the purified water reservoir 42 to the specified target value. In at least one other embodiment, the heating element 22 operates to maintain a set water temperature for a period of time set by a user. In at least one such embodiment, the maintained temperature of the water in the purified water reservoir 42 ranges between 80-95 degrees Fahrenheit. In another such embodiment of this aspect, the temperature of the water in the purified water reservoir 42 is maintained at a desired comfort temperature as set by the user.

As further shown in FIG. 4, the control panel 32 further includes an input unit 68 for inputting a preset activation and deactivation time to program the processor 28 which has an internal clock connected to the current time display 66. The processor 28 activates the heating element 22 and the cooling element 24 when the input unit 68 activation time coincides with the current time display 66. Further, the processor 28 deactivates the heating element 22 and the cooling element 24 when the input unit 68 deactivation time coincides with the current time display 66. In another embodiment, the processor 28 activates the pump 26 used to filter water in the water reservoir 38 through the filter 34 to the purified water reservoir 42 when the input unit 68 activation time coincides with the current time display 66. The processor 28 deactivates the pump 26 when the input unit 68 deactivation time coincides with the current time display 66 and/or when water in the water reservoir 38 is depleted.

In another embodiment (not illustrated), a nasal irrigation solution preparation system further includes a remote control system, at least a receiver and controller portion of which is incorporated into the solution preparation unit. The remote control system may further incorporate a conventional tablet or smart-phone, which may include a dedicated application for such purpose. The remote control system may include a companion control device, separate from the solution preparation unit, that is configured for remote communication and providing command options for a user desired interaction with the processor 28. The processor 28 upon user direction controls at least one of the on-control 56, the off-control 58, the heating element 22, the cooling element 24, the warming-cooling plate 12, the start control 54, the input unit 68, and/or the pump 26.

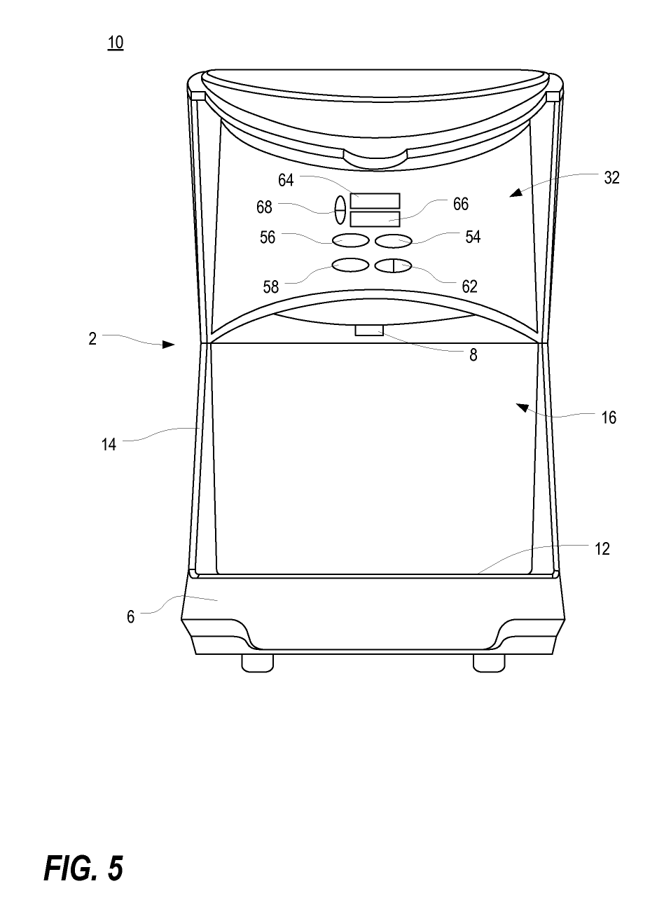

FIG. 5 is a front view of the nasal irrigation system 10 of FIG. 1 shown with the dispensing container 4 removed from the solution preparation unit 2. As will be appreciated, the dispensing container 4 is removable from the solution preparation unit 2, so that the dispensing container 4 may be used for rinsing the nasal and sinus passages and may be cleaned.

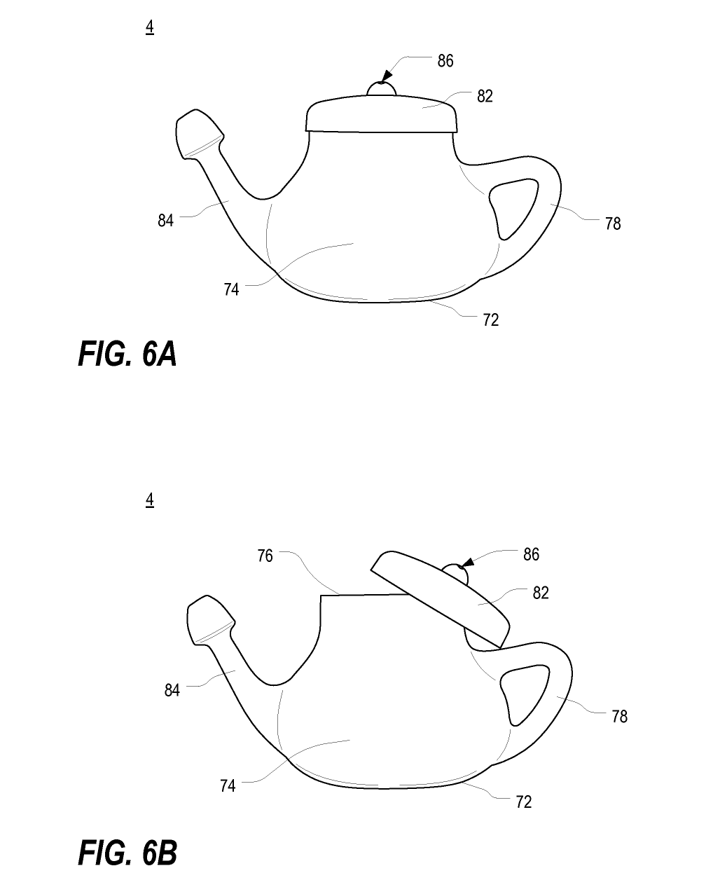

FIGS. 6A and 6B are side views of the dispensing container 4 of FIG. 1. As shown therein, the dispensing container 4 includes a main body 74 having a body 72, a rim 76, a handle 78, a lid 82, and an extending spout 84 which will fit snugly into a nostril to deliver the liquid solution contained therein to a user's sinus cavities. In FIG. 6A, the lid 82 is attached to the dispensing container 4, while in FIG. 6B the lid 82 is partially detached. The lid 82 has a circular rim which fits over, and is removable and replaceable on, the rim 76 of the dispensing container 4. The lid 82 features an opening 86 to facilitate the passage of filtered water and irrigant compounds released from the non-iodized salt reservoir 36, the purified water reservoir 42, and the pH buffer reservoir 44 into the dispensing container 4. In at least one embodiment, the rim 76 has an exterior circumventing exterior thread and the lid 82 has an interior thread matching the thread about the rim 76 to secure the lid 82 to the dispensing container 4. As will be appreciated, the dispensing container 4 is removable from within the solution preparation unit 2 as illustrated in FIG. 4.

In use, a user fills the non-iodized salt reservoir 36 by pouring non-iodized salt into the non-iodized salt reservoir 36. In another embodiment, the non-iodized salt reservoir 36 is detached from the solution preparation unit 2, filled with non-iodized salt, and then re-attached to the solution preparation unit 2. In another embodiment, an irrigant compound other than non-iodized salt is poured into the non-iodized salt reservoir 36. In another embodiment, one or more other medicinal or therapeutic material, such as an allergenic, analgesic, and/or antibiotic material, is poured into the non-iodized salt reservoir 36. In other embodiments, non-iodized salt and a mixture such as eucalyptus, chamomile, and/or one or more other medicinal or therapeutic material, such as an allergenic, analgesic, and/or antibiotic material, are poured into the non-iodized salt reservoir 36.

A user may also fill the pH buffer reservoir 44 by pouring sodium bicarbonate into the pH buffer reservoir 44. In another embodiment, the pH buffer reservoir 44 is detached from the solution preparation unit 2, filled with sodium bicarbonate, and then re-attached to the solution preparation unit 2. In another embodiment, an irrigant compound other than sodium bicarbonate is poured into the pH buffer reservoir 44. In another embodiment, one or more other medicinal or therapeutic material, such as an allergenic, analgesic, and/or antibiotic material, is poured into the pH buffer reservoir 44. In other embodiments, sodium bicarbonate and a mixture such as eucalyptus, chamomile, and/or one or more other medicinal or therapeutic material, such as an allergenic, analgesic, and/or antibiotic material, are poured into the pH buffer reservoir 44.

A user may also fill the tap water reservoir 38. This may be accomplished by detaching the water reservoir 38 from the solution preparation unit 2, filling the tap water reservoir 38 with water, and then re-attaching the water reservoir 38 to the solution preparation unit 2. In one method of use, a user may fill the tap water reservoir 38 by first filling the dispensing container 4 with a measured amount and thereafter pouring water from the dispensing container 4 into the water reservoir 38. The dispensing container 4 is then placed on the base 6 beneath the filler port 8. The dispensing container 4 may be provided with graded measurements for this purpose. In one contemplated commercial embodiment, the recommended quantity of water is seventeen to eighteen ounces, and the reservoirs 38,42 and dispensing container 4 are volumetrically sized accordingly.

Once the tap water reservoir 38 is filled, the water therein may then be filtered through the filter 34 having a pore size of one-micron or less to the purified water reservoir 42. In at least one embodiment, the on-control 56 activates the pump 26 which filters water in the tap water reservoir 38 through the filter 34 to the purified water reservoir 42. In another embodiment, water in the water reservoir 38 flows through the filter 34 to the purified water reservoir 42 via gravitational pull. In another embodiment, water in the tap water reservoir 38 flows directly to the purified water reservoir 42 without being filtered by the filter 34.

In at least some embodiments, water in the purified water reservoir 42 may be heated to a boil for at least three minutes to reduce or preferably eliminate the presence of microbial agents in the filtered water. A user may program the control panel 32 so that the water in the purified water reservoir 42 is boiled for a period greater than three minutes. Subsequently, water in the purified water reservoir 42 is cooled to a user selected comfort temperature. The user adjusts the temperature of the water in the purified water reservoir 42 by the temperature adjustment control 62 to a target temperature value. In other embodiments, water filtered through the filter 34 to the purified water reservoir 42 is dispensed into the dispensing container 4 without being heated. In still other embodiments, water in the purified water reservoir 42 may either be filtered or heated to a boil.

Activation of the start control 54 concurrently releases the optimal dose of non-iodized salt, filtered water, and sodium bicarbonate from the respective non-iodized salt reservoir 36, the purified water reservoir 42, and the pH buffer reservoir 44, through the respective passages or tubes 46,48,52 and the filler port 8 into the dispensing container 4. In another embodiment, the concurrent release of non-iodized salt, filtered water, and sodium bicarbonate from the non-iodized salt reservoir 36, the purified water reservoir 42, and the pH buffer reservoir 44 is carried out in response to preset activation. In another embodiment, the concurrent release of non-iodized salt, filtered water, and sodium bicarbonate from the non-iodized salt reservoir 36, the purified water reservoir 42, and the pH buffer reservoir 44 is carried out in response to activation by a remote communication command.

With the dispensing container 4 ready for use, the user may remove the dispensing container 4 from the base 6 using the handle 78. The user may then hold the dispensing container 4 so that the extending spout 84 fits into a nostril of the person whose nasal and sinus passages or cavities are to be rinsed. The dispensing container 4 is then tilted to gently force the solution of one or more of non-iodized salt, filtered water, and sodium bicarbonate to enter the user's nostril and sinus cavity. The user controls the flow of solution by the tilt of the dispensing container 4. The user repeats the process to the other nostril. The user preferably allows the solution in the nasal and sinus passages or cavities to drain. To help facilitate solution drainage from the nostril and sinus cavity the patient may gently blow the nose. The remaining solution in the dispensing container 4 may then be discarded and the dispensing container 4 may be cleaned.

Based on the foregoing information, it will be readily understood by those persons skilled in the art that the present invention is susceptible of broad utility and application. Many embodiments and adaptations of the present invention other than those specifically described herein, as well as many variations, modifications, and equivalent arrangements, will be apparent from or reasonably suggested by the present invention and the foregoing descriptions thereof, without departing from the substance or scope of the present invention.

Accordingly, while the present invention has been described herein in detail in relation to one or more preferred embodiments, it is to be understood that this disclosure is only illustrative and exemplary of the present invention and is made merely for the purpose of providing a full and enabling disclosure of the invention. The foregoing disclosure is not intended to be construed to limit the present invention or otherwise exclude any such other embodiments, adaptations, variations, modifications or equivalent arrangements; the present invention being limited only by the claims appended hereto and the equivalents thereof.

1. A nasal irrigation system for rinsing nasal and sinus passages or cavities, comprising:
a solution preparation unit, including a purified water reservoir, a base, and a filler port disposed above and over the base, that dispenses purified water through the filler port for use in a nasal rinse solution; and
a dispensing container that receives the nasal rinse solution, while positioned on the base, from the filler port of the solution preparation unit, and that administers the nasal rinse solution, while removed from the base, to a user via a direct nostril interface.

2. The nasal irrigation system of claim 1, wherein the purified water reservoir is disposed within a housing that is supported by above the base.

3. The nasal irrigation system of claim 2, further comprising a control circuit.

4. The nasal irrigation system of claim 3, further comprising a second water reservoir and a filter, the second water reservoir being in fluid communication with the filter and the purified water reservoir such that water contained in the second water reservoir flows from an outlet, through the filter, and into the purified water reservoir.

5. The nasal irrigation system of claim 4, wherein the filter is a particle filter having a pore size of one-micron or less.

6. The nasal irrigation system of claim 3, further comprising a heating element, under the control of the control circuit, that is in thermal contact with the purified water reservoir.

7. The nasal irrigation system of claim 6, wherein the heating element is adapted to boil water contained in the purified water reservoir.

8. The nasal irrigation system of claim 6, wherein the heating element is adapted to maintain water contained in the purified water reservoir at a temperature of 85-90 degrees Fahrenheit.

9. A nasal irrigation system for rinsing nasal and sinus passages or cavities, comprising:
a solution preparation unit, including:
a first water reservoir,
a filter that is in fluid communication with an outlet of the first water reservoir and filters water passing therethrough,
a second water reservoir that is in fluid communication with an outlet of the filter,
a heating element that brings filtered water in the second water reservoir to a boil to disinfect and sterilize the filtered water, and
a filler port; and
a dispensing container that receives a nasal rinse solution from the solution preparation unit, via the filler port, and administers the nasal rinse solution to a user via a direct nostril interface.

10. The nasal irrigation system of claim 9, further comprising a control circuit.

11. The nasal irrigation system of claim 10, further comprising a cooling element that cools heated water in the second water reservoir to a set temperature under the control of the control circuit.

12. The nasal irrigation system of claim 11, wherein the cooling element cools heated water in the second water reservoir to a temperature set by a user.

13. The nasal irrigation system of claim 10, wherein the heating element maintains water contained in the second water reservoir at a set temperature under the control of the control circuit.

14. The nasal irrigation system of claim 13, wherein the heating element maintains water contained in the second water reservoir at a temperature set by a user.

15. The nasal irrigation system of claim 13, wherein the heating element maintains water contained in the second water reservoir at a temperature of 85-90 degrees Fahrenheit.

16. The nasal irrigation system of claim 10, further comprising a pump that forces water from the first water reservoir through the filter to the second water reservoir.

17. The nasal irrigation system of claim 10, wherein the water flows from the first water reservoir through the filter to the second water reservoir via gravity flow.

18. A nasal irrigation system for rinsing nasal and sinus passages or cavities, comprising:
a solution preparation unit, including:
a water reservoir,
a device for purifying water in the water reservoir,
an additive reservoir, and
a filler port; and
a dispensing container that receives a nasal rinse solution from the solution preparation unit, via the filler port, and administers the nasal rinse solution to a user via a direct nostril interface;
wherein the nasal rinse solution is a combination of purified water from the water reservoir and an additive from the additive reservoir.

19. The nasal irrigation system of claim 18, further comprising a control circuit.

20. The nasal irrigation system of claim 19, wherein the device for purifying water is a particle filter that removes particles from water flowing into the water reservoir.

21. The nasal irrigation system of claim 19, wherein the device for purifying water is a heating element that brings water in the water reservoir to a boil.

22. The nasal irrigation system of claim 19, wherein the additive reservoir contains non-iodized salt to calibrate the salinity of the nasal rinse solution.

23. The nasal irrigation system of claim 19, wherein the additive reservoir contains a medicinal or therapeutic material.

24. The nasal irrigation system of claim 23, wherein the medicinal or therapeutic material is an allergenic, analgesic, and/or antibiotic material.

25. The nasal irrigation system of claim 19, wherein the additive reservoir contains a pH buffer.

26. The nasal irrigation system of claim 25, wherein the pH buffer is sodium bicarbonate.

27-71. (canceled)
